(19) United States
(12) Patent Application Publication
(10) Pub. No.: US 2025/0288233 A1
(43) Pub. Date: Sep. 18, 2025

(54) METHOD FOR DERIVING AND STORING EMOTIONAL CONDITIONS OF HUMANS

(71) Applicant: Social Health Innovations, Inc., Dover, DE (US)

(72) Inventors: Amanda Johnstone, South Launceston (AU); Roy Sugarman, Rose Bay (AU); Sean Ray Mullins, Darlinghurst (AU); Marcus Chidgey, London (GB); Zac Rowley, Terrigal (AU); James Keppel, Gledswood Hills (AU); Oliver Rozynski, Bomaderry (AU)

(21) Appl. No.: 19/071,670

(22) Filed: Mar. 5, 2025

Related U.S. Application Data
(63) Continuation of application No. 16/805,681, filed on Feb. 28, 2020, now abandoned.
(60) Provisional application No. 62/811,817, filed on Feb. 28, 2019.

(51) Int. Cl. A61B 5/16; A61B 5/00; A61B 5/021; G16H 10/60; G16H 20/70; G16H 50/20; G16H 50/30

(52) U.S. Cl. CPC A61B 5/165; A61B 5/0008; A61B 5/0048; A61B 5/021; A61B 5/6801; A61B 5/742; G16H 10/60; G16H 20/70; G16H 50/20; G16H 50/30; A61B 2562/02

(57) ABSTRACT

One variation of a method for deriving and storing emotional conditions of humans includes: writing timeseries biosignal data, output by a set of biosensors in a local device coupled to a user, to a rolling buffer spanning a look-back duration; in response to a trigger event at a first time, retrieving a set of biosignal data, spanning a first period of time preceding the first time, from the rolling buffer; transforming the set of biosignal data into a timeseries of emotions exhibited by the user during the first period of time; generating a visualization of the timeseries of emotions; and rendering the visualization of the timeseries of emotions on a display.

### CROSS-REFERENCE TO RELATED APPLICATIONS

This Application is a continuation of U.S. patent application Ser. No. 16/805,681, filed on 28 Feb. 2020, which claims the benefit of U.S. Provisional Application No. 62/811,817, filed on 28 Feb. 2019, each of which is incorporated in its entirety by this reference.

### TECHNICAL FIELD

This invention relates generally to the field of human psychophysiological events and more specifically to a new and useful method for deriving and storing emotional conditions of humans in the field of human psychophysiological events.

### BRIEF DESCRIPTION OF THE FIGURES

FIG. 1 is a flowchart representation of a method;

FIG. 2 is a flowchart representation of one variation of the method;

FIGS. 3A and 3B are flowchart representations of one variation of the method;

FIG. 4 is a flowchart representation of one variation of the method;

FIGS. 5A, 5B, and 5C are graphical representations of variations of the method; and

FIG. 6 is a flowchart representation of one variation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

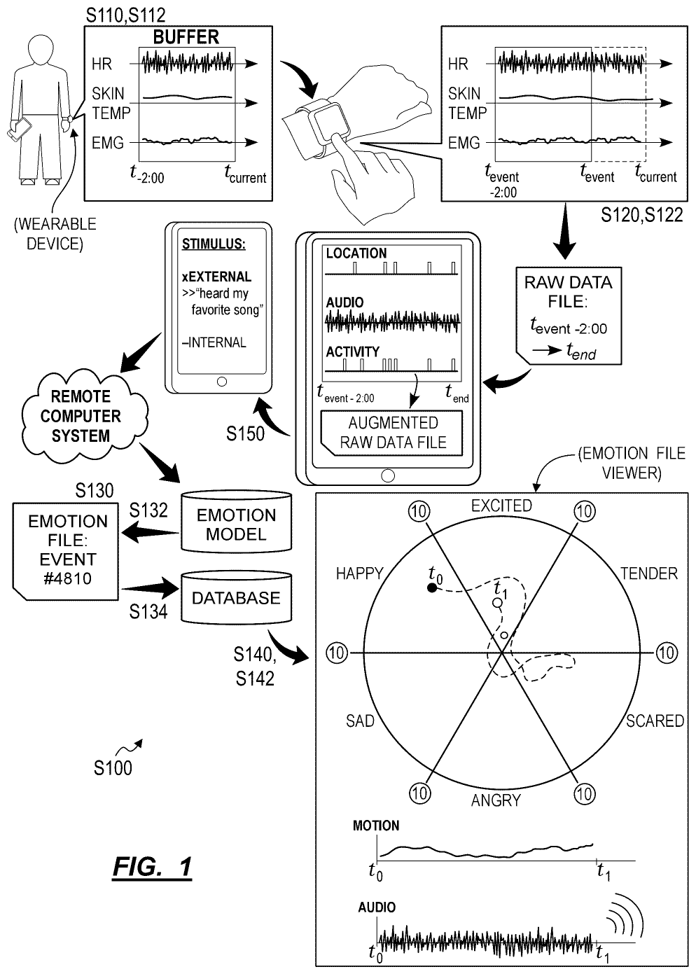

As shown in FIG. 1, a method S100 for deriving and storing emotional conditions of humans includes, at a local device coupled to a user: sampling a set of biosensors in the local device in Block S110; writing timeseries biosignal data from the set of biosensors to a rolling buffer spanning a look-back duration in Block S112; and, in response to detecting a trigger event at a first time, writing timeseries biosignal data, contained in the buffer and spanning a first period of time preceding the first time, to a raw data file in Block S120 and writing timeseries biosignal data, spanning a second period of time succeeding the first time, to the raw data file in Block S122. The method S100 also includes: transforming timeseries biosignal data in the raw data file into a timeseries of emotions exhibited by the user in Block S130; storing the timeseries of emotions in an emotion file in Block S132; and replaying a visualization of the timeseries of emotions in the emotion file in Block S142.

A similar variation of the method S100 shown in FIG. 1 includes, at a local device coupled to a user: writing timeseries biosignal data, output by a set of biosensors in a local device coupled to a user, to a rolling buffer spanning a look-back duration in Block S112; and, in response to a trigger event at a first time, writing timeseries biosignal data, contained in the rolling buffer and spanning a first period of time preceding the first time, to a raw data file in Block S120 and writing timeseries biosignal data, spanning a second period of time succeeding the first time, to the raw data file in Block S122. In this variation, the method S100 also includes: transforming timeseries biosignal data in the raw data file into a timeseries of emotions exhibited by the user in Block S130; generating a visualization of the timeseries of emotions in Block S140; and rendering the visualization of the timeseries of emotions on a display in Block S142.

Another variation of the method S100 shown in FIG. 1 includes: writing timeseries biosignal data, output by a set of biosensors in a local device coupled to a user, to a rolling buffer spanning a look-back duration in Block S112; in response to a trigger event at a first time, retrieving a set of biosignal data, spanning a first period of time preceding the first time, from the rolling buffer in Block S120; transforming the set of biosignal data into a timeseries of emotions exhibited by the user during the first period of time in Block S130; generating a visualization of the timeseries of emotions in Block S140; and rendering the visualization of the timeseries of emotions on a display in Block S142.

A similar variation of the method S100 shown in FIG. 1 includes: writing timeseries biosignal data, output by a set of biosensors in a local device coupled to a user, to a rolling buffer spanning a look-back duration in Block S112; in response to a trigger event at a first time, retrieving a set of biosignal data, spanning a first period of time preceding the first time, from the rolling buffer in Block S120; transforming the set of biosignal data into a timeseries of emotions exhibited by the user during the first period of time in Block S130; prompting the user to indicate an external stimulus that initiated the change in emotional state of the user in Block S150; storing the timeseries of emotions, labeled with the external stimulus identified by the user, in an emotion file associated with the user in Block S132; and storing the emotion file in a database in Block S134. This variation of the method S100 also includes, at a second time succeeding the first time: receiving selection of the emotion file in Block S160; generating a visualization of the external stimulus and the timeseries of emotions stored the emotion file Block S140; and rendering the visualization of the timeseries of emotions on a display in Block S142.

### 2. Applications

Generally, the method S100 can be executed by a system-such as including a wrist, head, or neck-born wearable device containing biosensors and worn by a user, a mobile device carried by a user, an implantable device containing biosensors, earbuds containing biosensors, and/or a remote computer system (e.g., a remote server, a computer network)—to: collect timeseries biosignal data of the user during an “emotion event”; interpret emotions and/or emotion intensities of the user during the emotion event from these biological data; and store a timeseries of these derived emotions and/or emotion intensities of the user in a format that can be transformed into a visualization that can be replayed at a later time and/or in real-time, such as to enable the user to review past personal emotional experiences and to share these personal emotional experiences in a visual format with others (e.g., friends, family, a therapist, followers)

In particular, a wearable device can store raw biosignal data-collected via a set of integrated sensors—in a rolling buffer. While or after the user experiences an internal or external stimulus that effects an emotion change, the user may decide to capture a record of this emotion change, such as for personal reference or to share with others. Accordingly, the user may manually select a trigger on her mobile device (e.g., a smartphone), which triggers her mobile device to: retrieve contents of the buffer; store these contents in a raw data file; interpret a sequence of emotion and/or emotion magnitude changes from these raw data; store these emotions in an emotion file; compile these emotions into a visualization depicting changes in the user's emotional state during this experience; and to upload this raw data file or an emoiton file depicting this sequence of emotion and/or emotion magnitude changes to a remote server for storage. For example, the mobile device can generate an emotion file that can be replayed in a visualizer in order to enable the user (or other human) to “see” the user's emotions and personal experience during or responsive to this internal or external stimulus.

The user may also label the raw data file and/or the emotion file with a descriptor of the experience or the internal or external stimulus and elect to store these data for later access. The mobile device can upload the raw data file and/or the emotion file-labeled accordingly and with additional time, date, and/or location metadata—to a remote database for storage. Later, the mobile device can retrieve this raw data file and/or the emotion file and render a visualization of the user's emotional state-responsive to the earlier internal or external stimulus-when labels stored with this raw data file and/or the emotion file match search terms entered by the user.

Additionally or alternatively, the user may elect to send this raw data file and/or the emotion file to a recipient, and the recipient's mobile device can similarly generate and render this visualization of the user's emotional state responsive to the internal or external stimulus, thereby providing the recipient greater insight into how the user responds to stimuli. “emotion” is described herein as a measurable (or “quantifiable”) physiological or psychophysiological event experienced by a human—such as perceived by the human as an emotive event—and that represents immediate or delayed responses to external stimuli or internal sensory effects.

### 2.1 Examples

For example, a wearable device (or a “local device”) worn on a user's wrist (e.g., a smartwatch) or neck (e.g., a “smart” pendant) can write biosignal, audio, location, and/or motion data to a two-minute buffer; the user may trigger the wearable device to store contents of the buffer and subsequent biosignal and environmental data in a raw data file when the user experiences a stressful event, such as being harassed on a street, being reprimanded by a superior at work, or getting a call from a teacher at her son's school. In this example, the wearable device can offload these data to the user's mobile device (e.g., smartphone) or to the remote computer system (e.g., via the mobile device), which can then implement an emotion interpretation model to transform these timeseries biosignal data into emotions and emotion intensities and compile these derived emotion, user motion, location, and/or environment data into an emotion file. The user may then share this emotion file with her therapist or other interested party, who may view a visualization of the emotion event stored in the emotion file in order to gain better insight into the user's emotions and psychophysiological response during or after the emotion event.

In another example, the user may trigger the wearable device to store contents of the buffer and subsequent biosignal and environmental data in a raw data file at the beginning of the user's wedding ceremony. In this example, the wearable device can offload these data to the user's mobile device (e.g., smartphone) or to the remote computer system (e.g., via the mobile device) when conclusion of the emotion event is indicated manually by the user. The user's mobile device or the remote computer system can then implement an emotion interpretation model to transform these timeseries biosignal data into emotions and emotion intensities and compile these derived emotion, user motion, location, and/or environment data into an emotion file. The user may later share this emotion file with her children or privately view a visualization of the emotion event stored in the emotion file—such as paired and synchronized to a video of the wedding ceremony—in order to more accurately recollect her wedding ceremony.

In yet another example, the user may trigger the wearable device to store biosignal and environmental data in a raw data file at the beginning of a music recording session in a recording studio. In this example, the wearable device can offload these data to the user's mobile device (e.g., smartphone) or to the remote computer system (e.g., via the mobile device), which can then implement an emotion interpretation model to transform these timeseries biosignal data into emotions and emotion intensities and compile these derived emotion, user motion, location, and/or environment data into an emotion file. The user, a sound technician, or a producer, etc. may then: synchronize a song produced during the music recording session to a concurrent timeseries of emotions and emotion intensities stored in the emotion file; and combine the song and timeseries emotions and emotion intensities into a multimedia song file. When this multimedia song file is later replayed by a follower of the user at another computing device, the computing device can render a visualization of the user's emotions and emotion intensities while recording the song—stored in the multimedia song file—synchronized to the audio track.

In another example, a therapist or medical personnel accesses emotional data recorded by a subject's wearable device during an intervention for the subject in order to measure the subject's neurohumeral or physiological response to the intervention and thus inform future treatment for the subject.

Generally, an In yet another example, upon receiving and reading a text message—received from a sender—within a messaging application executing on a mobile device (e.g., a smartphone, a tablet) and then experiencing an emotional response to this text message, the user may select an option within the messaging application to share this emotional response with the sender. Accordingly, the messaging application (or other native application executing on the mobile device) can execute Blocks of the method S100 to: transmit a query for buffer contents to a wearable device worn by the user; download raw biosignal data returned by the wearable device; interpret a sequence of emotions represented in these raw biosignal data; and generate and render a visualization of this sequence of emotions within an emotion viewer. The messaging application (or other native application) can then: prompt the user to trim the visualization to a period time of concurrent with the user's consumption of the text message; trim the visualization accordingly; and transmit this trimmed visualization to the sender once confirmed by the user. The messaging application (or other native application) can therefore execute Blocks of the method S100 to enable the user to show (e.g., “expose through visualization”) how reading the text message from the sender effected a change in the user's emotions, which may be more powerful and authentic than the user merely telling the sender about these emotions in a text message response.

In a similar example, while or after composing a message for a recipient within a messaging application executing on a computing device, the user may elect an option within the messaging application to share her emotional response while drafting the message with the recipient. Accordingly, the messaging application (or other native application executing on the computing device) can execute Blocks of the method S100 to: transmit a query for buffer contents to a wearable device worn by the user; download raw biosignal data returned by the wearable device; interpret a sequence of emotions represented in these raw biosignal data; and generate and render a visualization of this sequence of emotions within an emotion viewer. The messaging application (or other native application) can then: prompt the user to trim the visualization to a period time of concurrent with the user's composition of the text message; trim the visualization accordingly; and transmit this trimmed visualization to the recipient once confirmed by the user. The messaging application (or other native application) can therefore execute Blocks of the method S100 to enable the user to show how composing the text message for the recipient effected a change in the user's emotions, which may be more powerful and authentic than the user merely telling the recipient the intent of her text message.

### 2.2 Post Hoc Emotion Recordation

Therefore, because the wearable device captures user biosignal data-representative of the user's emotions-automatically and continuously throughout operation, the system can enable the user to retrieve a record of her emotions after a recent “emotion event” a) without interrupting this experience to initiate recordation of her emotions and b) without requiring the user to predict upcoming emotion events. The system can then: generate a raw data file and/or an emotion file representing the user' emotions during this emotion after based on later manual confirmation of this emotion event; generate a visualization of these emotions; and present this visualization to the user, a friend, a family member, a coworker, an acquaintance, and/or a therapist, thereby enabling the user to observe and share her emotional experience during this emotion event.

Furthermore, because the computer system writes these biosignal data to a buffer of limited duration (e.g., two minutes, five minutes) and discards biosignal data older than this limited duration if not otherwise recalled responsive to confirmation (or detection) of an emotion event, the computer system can efficiently allocate data storage to biosignal and/or emotion data representative of emotion events of interest to the user.

### 3. System

As described above, Blocks of the method S100 can be executed by a wearable device (or “local device”) worn by a user, a mobile device carried by the user, and/or a remote computer system (e.g., a computer network, a remote server).

Generally, the wearable device is configured to collect timeseries biosignal, motion, and/or environmental data while worn by a user. For example, the wearable device can be configured to be worn on a user's wrist (e.g., in the form of a smartwatch), ankle, or belt. In another example, the wearable device includes a set of headphones, an ear bud, or a pair of smart glasses. The wearable device can also include multiple biosensors, such as: a heart rate sensor; a skin temperature sensor; an EEG sensor; an EMG sensor; and/or a galvanic skin response sensor. The wearable device can also include: a motion sensor, such as an accelerometer, gyroscope, and/or compass sensor; a microphone (e.g., to record an audio stream); a location sensor (such as a GPS sensor configured to detect the wearable device's geospatial location); a buffer (e.g., a two-minute rolling buffer); longer-term data storage (or “memory”); and/or a user interface, such as a physical pushbutton or voice-control functions to trigger or end an emotion event.

For example, the wearable device can write timeseries biosignal data-output by a heart rate sensor, a skin temperature sensor, and a galvanic skin response sensor integrated into the wearable device while worn by the user—to a rolling buffer in local memory. In this example, the wearable device can implement: a preset, fixed look-back duration (e.g., approximately five minutes, such as between three and six minutes); or a user-defined, location-based, time-based, or contextual look-back duration, such as two minutes on weeknights, three minutes on weekends, four minutes when occupying a workplace, and ten minutes when recording media (e.g., audio or video) on a connected mobile device.

The wearable device can further include a wireless communication module: configured to send contents of the buffer and/or saved data to the user's mobile device, such as after a “save trigger” and up to an “end trigger” entered by the user via the user interface; and/or configured to stream data to the mobile device after selection of the save trigger and until selection of the end trigger by the user. The wearable device can also: detect proximity of other devices-such as mobile devices and wearable devices associated with other humans, spaces, locations, and/or or activities-via the wireless communication module; and write identifiers of these other humans, spaces, locations, and/or or activities to the buffer or local memory when these other devices are detected. The wearable device can also: maintain an internal timer; intermittently synchronize the internal timer to the user's mobile device via the wireless communication module; and timestamp biosignal and other data stored in the buffer and/or in a raw data file with time and date data from the internal timer.

The system can also include a native application configured to execute on the user's mobile device. Throughout operation, the native application can: collect data through sensors in the mobile device (e.g., location, motion, and manual selection of a virtual “save trigger” and/or a virtual “end trigger” rendered on a display of the mobile device); and download raw data files from the wearable device via a wireless connection; and implement methods and techniques described below to transform these data into timeseries emotions of the user and/or upload the raw data files to the remote computer system for remote processing.

The system can additionally or alternatively include a remote computer system configured to remotely process raw biosignal data collected by the wearable device (and/or by the mobile device) to derive timeseries of user emotions and to generate emotion files depicting user emotions during emotion events. The remote computer system (or the wearable device, the mobile device) can also develop an emotion interpretation model that links biosignal values to certain emotions and/or emotion intensities of the user and/or develop an emotion interpretation model that links certain wearable device or mobile device outputs, user actions, user experiences, etc. to changes in emotions and/or emotion intensities for the user based on data collected by the wearable device and/or the mobile device over time.

### 4. Timeseries Data Collection

Blocks S110 and S112 of the method S100 recite, at a local device coupled to a user, sampling a set of biosensors in the local device and writing timeseries biosignal data from the set of biosensors to a rolling buffer spanning a look-back duration. Blocks S120 and S122 of the method S100 recite, in response to detecting a trigger event at a first time: writing timeseries biosignal data, contained in the buffer and spanning a first period of time preceding the first time, to a raw data file; and writing timeseries biosignal data, spanning a second period of time succeeding the first time, to the raw data file. Generally, in Blocks S110, S112, S120, and S122, the wearable device writes biosignal, motion, and/or environmental data to a rolling buffer while in operation and selectively stores snippets of these data in “raw data files” responsive to receipt of a manual input indicating an emotion event and/or in response to automatically detecting an emotion event.

In one implementation, the wearable device: regularly samples values from its biosensors, such as at a rate of 10 Hz, in Block S110; writes these biosignal data—with timestamps—to a rolling buffer, such as spanning a duration of two minutes, in Block S112; and clears biosignal data older than this duration from the rolling buffer. The wearable device can also sample other sensors in the wearable device and write these other sensor data—such as the wearable device's location, ambient noise, and/or unique identifiers wirelessly broadcast by other devices nearby—to the rolling buffer in Block S112.

### 4.1 Manually Emotion Event Trigger at Wearable Device

In this implementation, when the user selects a save trigger input (or other user interface) on the wearable device, the wearable device can: preserve contents of the buffer, such as by moving current contents of the buffer to local memory in Block S120; and continue to sample these biosensors and other sensors in Block S122, as shown in FIG. 1. When the user later selects an end trigger input (or other user interface) or reselects the save trigger input on the wearable device, the wearable device can: cease storage of biosignal and other data; generate a raw data file containing these biosignal and other data spanning this emotion event (or “emotion period of interest”; and return to writing biosignal and other data to the buffer only. The wearable device can also transmit this raw data file to the user's mobile device, such as immediately or as soon as a wireless connection to the mobile device is established.

In this foregoing implementation, the mobile device can also write data to a second rolling buffer throughout operation, such as motion data, location data, audio data, and/or wireless connectivity data to other devices nearby. In this implementation, when the user selects the save trigger input at the wearable device, the wearable device can also broadcast a save trigger to the mobile device. Upon receipt of this save trigger, the mobile device can similarly preserve contents of the second buffer and continue to generate and store timestamped data from these sensors to local memory. When the user later selects the end trigger input or reselects the save trigger input at the wearable device, the wearable device can broadcast an end trigger to the mobile device. Accordingly, the mobile device can: cease storage of sensor data; generate a second raw data file containing these data and spanning the emotion event; and return to writing data from these sensors to the second buffer. Upon receipt of the raw data file from the wearable device, the mobile device can merge and synchronize data in the raw data file and the second raw data file into an augmented raw data file spanning this emotion event. The mobile device can then process this augmented raw data file locally or transmit this augmented raw data file to the remote computer system for processing, as described below.

### 4.3 Live Data Offload

In another implementation, when the user selects the save trigger input, the wearable device can: confirm or establish a wireless connection with the mobile device; transmit contents of the buffer to the mobile device; and then stream subsequent timestamped biosignal and other data to the mobile device until the user selects the end trigger input. In this implementation, upon receipt of these data from the wearable device, the mobile device can also: generate timestamped motion, location, audio, wireless connectivity, and/or other data; aggregate data generated locally and received from the wearable device; and save these data in one augmented raw data file spanning the emotion event.

### 4.4 User Activity

In the foregoing implementations, the wearable device can also implement action or activity recognition techniques to transform raw motion data into actions or activities performed by the user and write a timeseries of actions or activities to the buffer and/or to the raw data file. Additionally or alternatively, the mobile device can implement activity recognition techniques to transform raw motion data collected by the mobile device, raw motion data received from the wearable device, and/or interpreted actions or activities received from the wearable device into a timeseries of actions or activities performed by the user between selection of the save and end trigger inputs by the user.

### 4.5 Raw Data Storage

In one implementation, the mobile device stores the raw data file or the augmented raw data file (hereinafter the “raw data file”) in local memory until released by the user for emotion interpretation and other processing.

### 4.6 Manual Trigger at Mobile Device

In one variation, the mobile device renders a virtual emotion capture button within: a native text messaging, email, camera, or health application executing on the mobile device; or on a home screen or within a home menu rendered on the mobile device. In this variation, when the user selects this virtual emotion capture button at the mobile device, the mobile device can: transmit a query to the wearable device for current contents of the rolling buffer; and write data received from the wearable device to a raw data file stored in local memory on the mobile device.

Furthermore, in the variation, if the user elects to continue emotion recordation after selection of the virtual emotion capture button or if the mobile device is currently configured to continue recordation of raw biosignal data following selecting of the virtual emotion capture button, the mobile device can: serve a command to the wearable device to stream biosignal data captured by sensors in the wearable device back to the mobile device; and append these biosignal data to the raw data file for this emotion event. For example, if the user taps the virtual emotion capture button at the mobile device once, the mobile device can transmit a command to the wearable device to return the current contents of the rolling buffer only. However, if the user double-taps the virtual emotion capture button at the mobile device, the mobile device can transmit a command to the wearable device to both return the current contents of the rolling buffer and to stream subsequent biosignal data back to the mobile device until the user again taps the virtual emotion capture button at the mobile device.

### 5. Emotion Detection

Block S130 of the method S100 recites transforming timeseries biosignal data in the raw data file into a timeseries of emotions exhibited by the user; and Block S132 of the method S100 recites storing the timeseries of emotions in an emotion file. Generally, in Blocks S130 and S132, the remote computer system can: derive emotions, emotion intensities, and/or emotion changes throughout an emotion event based on biosignal data contained in the raw (and annotated, augmented) data file; and then store a timeseries of these emotions, emotion intensities, and/or emotion changes—synchronized to other location, motion, activity, ambient noise, and/or other sensor data—in an emotion file.

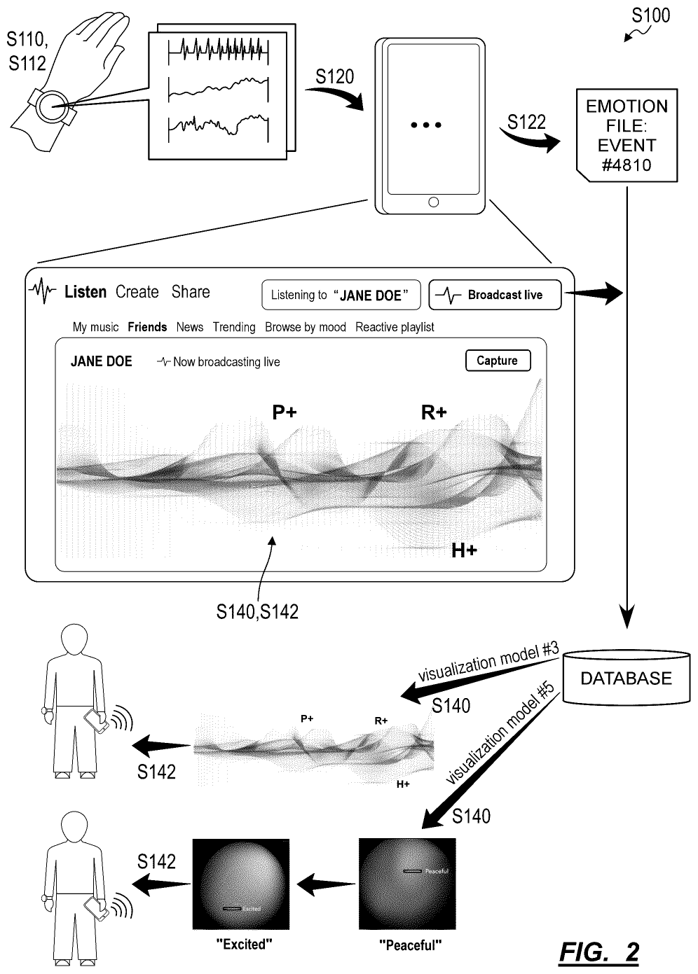

In one implementation, the mobile device uploads the raw data file to the remote computer system for remote, asynchronous processing; and the remote computer system then implements an emotion interpretation model to transform timeseries biosignal data into a timeseries of emotions and emotion intensities based on a generic or customized emotion interpretation model, as shown in FIG. 2. For example, the emotion interpretation model can define a parametric model that outputs quantitative values representative of emotions and emotion intensities based on discrete or timeseries heart rate, heart rate variability, skin temperature, galvanic skin response, and/or other biosignal values. Alternatively, the emotion interpretation model can include a neural network or other deep learning or machine learning model that ingests timeseries biosignal data and outputs timeseries emotion and emotion intensity values paired with confidence scores for these values.

In the foregoing implementation, during a setup period or early user period over several days or weeks, the mobile device (or the wearable device) regularly prompts the user to manually indicate her current emotion state and/or to manually annotate a raw data file with her emotions and emotion intensities as described above. In this example, the remote computer system then: derives correlations between biosignals, user emotions, and emotion intensities; and constructs an emotion interpretation model unique to the user. Similarly, the remote computer system can implement deep learning, machine learning, regression, or other methods and techniques to construct an emotion interpretation model representing a relationship between biosignals, emotions, and emotion intensities for the user. In another example, the remote computer system: initially implements a generic emotion interpretation model to interpret emotions and emotion intensities from timeseries biosignal data contained in a raw data file; detects differences between emotions derived by the generic emotion interpretation model and emotion annotation manually entered into raw data files by the user; and refines the generic emotion interpretation model to better reflect unique emotional responses of the user based on these differences.

However, the remote computer system can implement any other method or technique to extract or derive timeseries emotion values from the raw data file in Block S130. The remote computer system can then: synchronize these timeseries emotion values with location, motion, activity, ambient noise, identities of other people nearby, and/or other data contained in the raw data file; and merge these emotion and other data into one emotion file that therefore represents the user's emotions, the user's actions, and/or ambient conditions around the user throughout the emotion event.

### 5.1 Manual Emotion Annotation

In one variation, the mobile device (e.g., the native application): renders a visualization of timeseries data in the raw data file on the mobile device, such as a graphical representation of the user's heart rate, heart rate variability, skin temperature, motion magnitude, and/or ambient noise level, etc. over the emotion event; and prompts the user to manually annotate segments of the emotion event with emotions and/or emotion intensities experienced by the user. For example, the mobile device can: automatically identify significant changes in heart rate, heart rate variability, and/or skin temperature—depicted in the raw data file—that exceed threshold changes through the emotion event; isolate segments of the emotion event bounded by such significant biosignal changes; prompt the user to label each segment in the emotion event by selecting an emotion from a dropdown menu containing a prepopulated list of emotions; and prompt the user to indicate an emotion intensity in each of these segments, such as on a scale from “1” to “10.”

The mobile device can then: aggregate these manual feedback from the user into a timeseries of emotions and emotion intensities; and store these emotion data in the raw data file and/or store this timeseries of emotions and emotion intensities in a new emotion file for the emotion event.

### 6. Stimulus Annotation

In one variation shown in FIG. 1, the method S100 further includes Block S150, which recites prompting the user to indicate an external stimulus that initiated the change in emotional state of the user. Generally, in Block S150, the mobile device prompts the user to indicate an internal stimulus, an external stimulus, and/or other descriptors for the emotion event and then labels the raw data file and/or the emotion file with the user's response to this prompt. For example, after the user triggers the emotion event and then indicates conclusion of the emotion event, the mobile device can: render a text field; prompt the user to enter up to ten tags for the emotion event into the text field (e.g., “happy, surprised, received flowers, at work, John Smith”; “happy, laughing, funny cat video, alone at home”; “excited, energized, recorded song with band”; or “sad, frustrated, read the news”); and then write these tags to the raw data file and/or to the emotion file.

### 7. Variation: Real-time Emotion Detection

In one variation, the wearable device or the mobile device locally implements the emotion interpretation model to transform biosignal data recorded during an emotion event into timeseries emotions and emotion intensities, such as in (near) real-time.

In one example, the remote computer system, the mobile device, and the wearable device cooperate as described above: to collect a raw data file from the user's wearable device and/or mobile device; to collect annotations for the raw data file from the user at the mobile device; and to develop or refine a custom emotion interpretation model for the user based on biosignal data contained in this raw data file and annotations provided by the user. The remote computer system then uploads this custom emotion interpretation model to the mobile device. The mobile device: implements this custom emotion interpretation model to locally transform biosignal data inbound from the wearable device—responsive to a manual trigger from the user for a new emotion event—into a new timeseries of emotions exhibited by the user during this new emotion event; and stores this timeseries of emotions in a new emotion file.

Alternatively, the mobile device can upload this custom emotion interpretation model to the wearable device. The wearable device can then: implement this emotion interpretation model to locally interpret user emotions from real-time biosignal data throughout operation; and write these derived emotions to the rolling buffer—in addition to or instead of raw biosignal data—such as in the form of timestamped flags for types and magnitudes of user emotions. When triggered by the user, the wearable device can aggregate flags for types and magnitudes of user emotion—stored in the rolling buffer—into a new emotion file and then offload this new emotion file to the mobile device, which can then upload this new emotion file to the remote computer system (such as after interfacing with the user to trim this emotion file to a time period relevant to the corresponding emotion event).

The mobile device and/or the remote computer system can also annotate or tag this emotion file with a time, date, geospatial location, user identifier, identifiers of other humans nearby, emotion types represented in the emotion file, user activities during the emotion event, a title or other descriptor manually entered by the user, internal or external stimuli indicated by the user, and/or other metadata.

In a similar variation, a local device (e.g., the wearable device and/or the mobile device) and the remote computer system can cooperate to implement an experiential feedback-loop in which: the local device collects biosignal data; the remote computer system interprets emotional data of the use from these biosignal data; the local device outputs an audio/visual experience to the user based on these emotional data; and the local device and the remote computer system monitor changes in the user's emotional state during this audio/visual experience and adjust this audio/visual experience output accordingly.

### 8. Variation: Automatic Emotion Event Detection and Data Storage

Furthermore, in the foregoing variation in which the wearable device stores and implements the emotion interpretation model to process biosignal data in real-time, the wearable device can automatically store timeseries emotion, biosignal, location, and/or environmental data, etc. in response to detecting a particular emotion, a change in emotion, or a change in emotion intensity, etc. from these biosignal data.

### 8.1 Emotion State Change

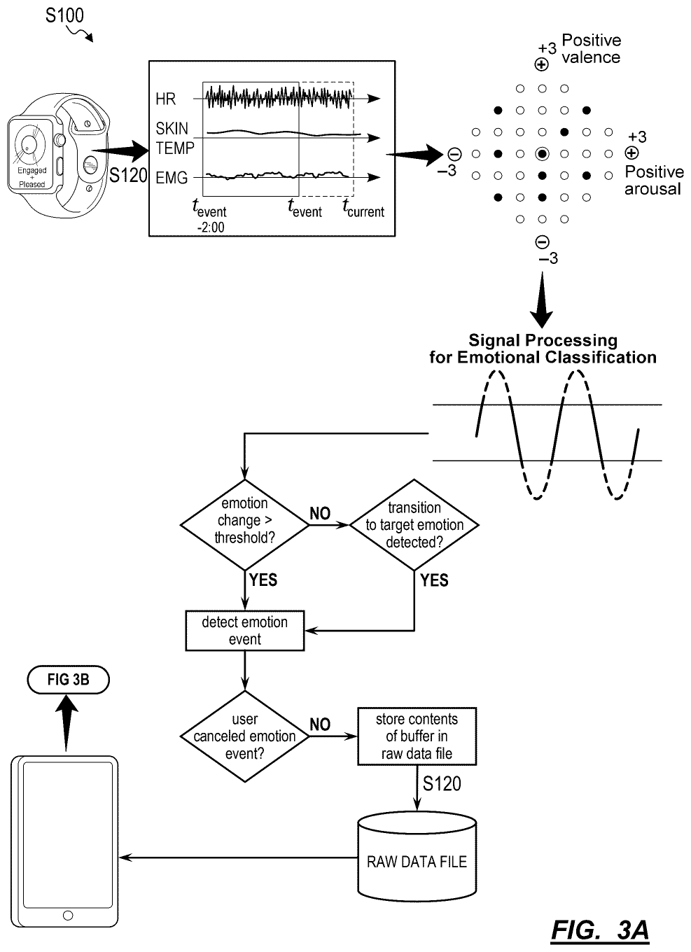

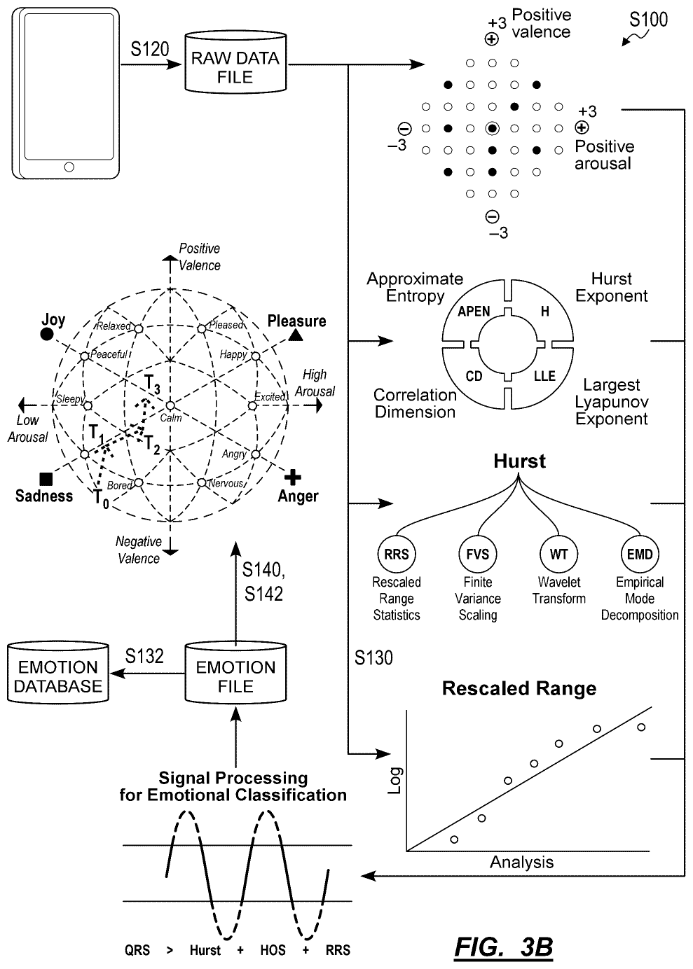

In one implementation shown in FIG. 3A, the wearable device can implement a first, small-footprint emotion interpretation model to detect user emotion changes based on heart rate and heart rate variability changes; and the mobile device or the remote computer system can implement a second, larger-footprint emotion interpretation model (e.g., a user-specific emotion interpretation model) to interpret emotions and emotion intensities from heart rate, heart rate variability, skin temperature, galvanic skin response, ambient noise, ECG, EEG, and/or other biosignal or environmental data. In this example, the wearable device can write biosignal and other data to the rolling buffer, as described above, and pass heart rate and heart rate variability data into the first emotion interpretation model to detect user emotion changes in real-time throughout operation. Then, in response to detecting an emotion change in the user—such as greater than a threshold amplitude change—the wearable device can indicate to the user that an emotion change was detected, such as by triggering a vibrator in the wearable device to pulse, by rendering a notification on a display on the wearable device, or by broadcasting a notification to the user's mobile device. The wearable device can then automatically write current contents of the buffer to memory and continue to write biosignal and other data read from sensors in the wearable device to longer-term memory. The wearable device can also cease recording biosignals during this emotion event at the earliest of: manual cancellation by the user via an input on the wearable device or mobile device; after a threshold period of time (e.g., two minutes) after the emotion event was detected; and reversal of the emotion change similarly detected by the wearable device. The wearable device can then package these raw data (and/or derived emotions, emotion changes) into a raw data file and serve this raw data file to the mobile device for processing and visualization.

Therefore, in the foregoing implementation, the wearable device can: implement a local (lower-resolution) emotion interpretation model to interpret emotional states of the user in near real-time based on biosignal data collected by the wearable device and stored in the local rolling buffer; interpret a change in emotional state of the user around a current time based on these biosignal data; retrieve a set of biosignal data—representing this change in emotional state and the emotional state of the user over a period of time leading up to this emotional change—from the rolling buffer in response to detecting this change in emotional state; write this set of biosignal data to a raw data file; and then offload this raw data file to the mobile device. Furthermore, the wearable device can stream subsequent biosignal data-following detection of this change in emotional state—to the mobile device, and the mobile device can write these subsequent biosignal data to the raw data file.

The wearable device can continue to stream these biosignal data to the mobile device and the mobile device can continue to write these biosignal data to the raw data file, such as until the user cancels or confirms recordation of this detected emotion event. For example, upon detection of this change in emotional state, the wearable device can output a soft vibration to alert the user of detection of the possible emotion event; and/or the mobile device can output a haptic, visual, and/or audible notification to prompt the user to confirm the emotion event. If the user then cancels or discards the possible emotion event, such as by silencing the wearable device or the mobile device, the wearable device can cease real-time broadcast of biosignal data to the mobile device, and the mobile device can discard the raw data file. However, if the user does not respond to the emotion event notification on the wearable device or the mobile device, the wearable device can continue to stream biosignal data to the mobile device, and the mobile device can continue to write these biosignal data to the raw data file until the earlier of: a preset maximum unconfirmed emotion event duration (e.g., two minutes) after detection of the emotional state change; and approximate return to the emotional state prior to the detected emotional state change. Yet alternatively, if the user confirms the emotion event notification, the wearable device can continue to stream biosignal data to the mobile device, and the mobile device can continue to write these biosignal data to the raw data file until the earlier of: a preset maximum confirmed emotion event duration (e.g., five minutes) after confirmation of the emotion event; and receipt of manual confirmation of the conclusion of the emotion event.

In the foregoing implementation, the mobile device or remote computer system can then: implement a second (higher-resolution) emotion interpretation model to interpret the user's emotions, emotion changes, and/or emotion intensities (e.g., with greater resolution and/or accuracy) from this raw data file; and compile these emotion data derived from the raw data file into an emotion file, as described above and shown in FIG. 3B.

Alternatively, after detecting the change in emotional state of the user, the wearable device can: compile emotion types and magnitudes from before and after the detected change in emotional state into an emotion file; implement the foregoing methods and techniques to prompt the user to confirm the possible emotion event; and then selectively upload this emotion file-rather than robotic raw biosignal data—to the mobile device.

The mobile device can then interface with the user as described above to annotate and/or tag the emotion file for the emotion event with an internal stimulus, an external stimulus, and/or other metadata.

### 8.2 Target Emotion Detected

In the forgoing implementation, the wearable device and/or the mobile device can automatically record a raw data file and/or an emotion file in response to detecting a user emotional state change into or out of (or toward or away from) a particular emotion type of interest previously specified by the user (and if a magnitude of this particular emotion type of interest is more or less than a threshold magnitude specified by the user).

For example, the mobile device or wearable device can record a particular emotion type of interest selected by the user for automatic emotion event capture, such as “anger” or “frustration”. Accordingly, the mobile device and/or the wearable device can automatically record a raw data file and/or an emotion file, as described above, in response to interpreting a transition into (or out of) an “anger” or “frustration” emotional state change. The mobile device can then prompt the user to indicate an internal or external stimulus that triggered this emotion event and label the emotion file for this emotion event accordingly. The mobile device can additionally or alternatively generate and render a visualization of emotions represented in this emotion file for the user, thereby enabling the user to visualize her progression from one emotional state to another around a time of the indicated stimulus.

### 9. Visualizations

Blocks S140 and S142 of the method S100 recite: generating a visualization of the timeseries of emotions; and rendering the visualization of the timeseries of emotions on a display. Generally, in Blocks S140 and S142, the user's mobile device or another computing device can later access and replay the emotion file, such as including rendering a graphical visualization of the user's emotions—synchronized to an audio track (e.g., ambient noise or post-processed audio)—during the emotion event.

In one implementation, the user's mobile device or other computing device executes an emotion file viewer. Upon receipt of a selection for an emotion file at the computing device, the emotion file viewer: accesses the emotion file; renders an emotion graph depicting multiple emotion axes (e.g., happy, excited, tender, scared, angry, sad); and initiates playback of the emotion file. During playback of the emotion file, the emotion file viewer can: render a pointer, avatar, or other emotional state indicator at a position on the graph corresponding to the derived emotion and emotion intensity indicated in the emotion file; and animate the pointer moving over the graph to depict emotion and emotion intensity changes identified in the emotion file.

For example, during playback of the emotion file, the emotion file viewer can render an emotion graph depicting a set of emotion axes and animate an avatar (e.g., an icon, temperature gradient) moving sequentially over regions of the emotion graph representing types and magnitude of the sequence of emotions specified in the emotion file, including: rendering the avatar at a first position on the emotion graph corresponding to a first emotion type at a first magnitude exhibited by the user at a first time during the emotion event; transitioning the avatar to a second position on the emotion graph corresponding to a second emotion type at a second magnitude exhibited by the user during a second time during the emotion event; and then transitioning the avatar to a third position on the emotion graph corresponding to the second emotion type at a third magnitude exhibited by the user at a third time during the emotion event, as shown in FIGS. 1 and 3B.

In another example, during playback of the emotion file, the emotion file viewer: renders a 2D or 3D anthropomorphic avatar with facial expressions and/or skin color (or “temperature”) depicting derived emotion and emotion intensity indicated in the emotion file; and animates the avatar performing motions, actions, and/or activities indicated in the emotion file. In this example, the emotion file viewer can render the user's avatar in a 2D or 3D virtual environment with virtual avatars of other people detected nearby during the emotion event, as recorded in the emotion file.

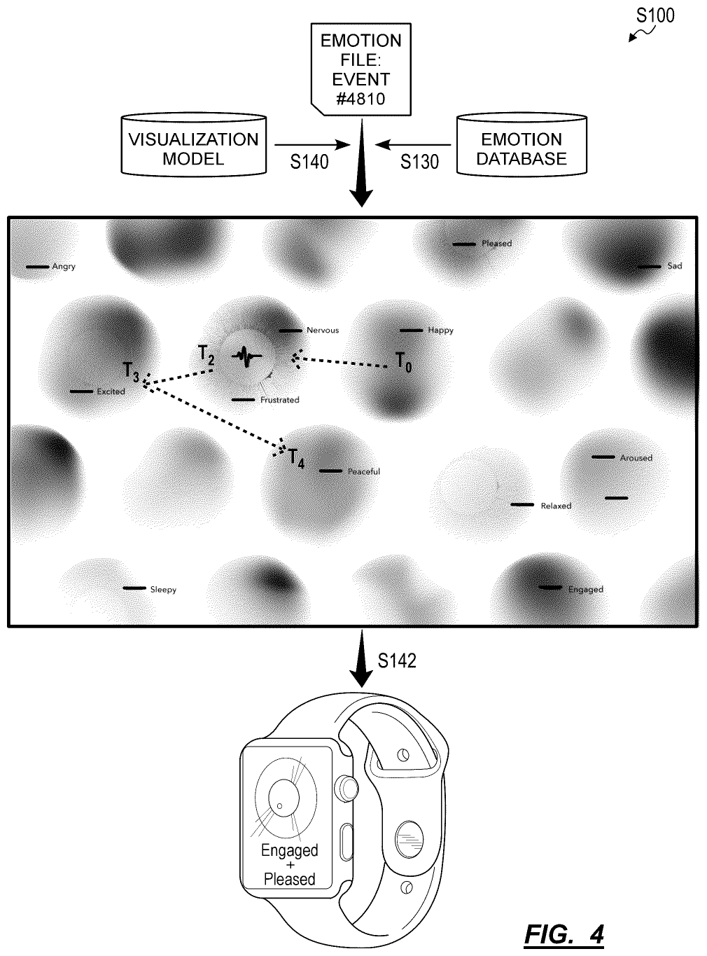

In yet another example shown in FIGS. 2 and 4, during playback of the emotion file, the emotion file viewer: renders a virtual ball of wax; morphs the color of the virtual ball of wax according to emotions indicated in the emotion file; adjusts an opacity of the virtual ball of wax according to emotion intensities indicated in the emotion file; and moves or deforms the virtual ball of wax proportional to motion or activity amplitude of the user during the emotion event, as depicted in the emotion file.

In another example shown in FIG. 2, the emotion file viewer can render an animated wave depicting a timeseries or “flow” of intensities of various emotion types, intensities, valence states, and/or arousal states represented in the emotion file.

In the foregoing examples, the emotion file viewer can also: replay an audio track (e.g., ambient noise) stored in the emotion file; and/or indicate a location of the emotion event, such as on a map or by rendering an address, building name, or space identifier stored in the emotion file. The emotion file viewer can additionally or alternatively render secondary graphs—synchronized to the emotion graph, virtual avatar, or virtual ball of wax—depicting motion magnitude, heart rate, heart rate variability, skin temperature, detected user actions or activities, and/or other raw or derived timeseries data during the emotion event and stored in the emotion file.

### 9.1 Visualization Options

In one variation, the system enables the user—and recipients of the user's emotion file—to select from a set of visualization types for the emotion file.

For example, following an emotion event, the user may elect to view an emotion timeseries for this emotion event—stored in a new emotion file—in a first visualization format and also send this new emotion file to a recipient (e.g., a friend, a therapist) in order to share this emotion event with the recipient. The recipient may prefer to view this emotion file in a second visualization format and thus elect this second visualization format-such as from a dropdown menu of available visualization formats-within an instance of the emotion file viewer executing on her computing device.

Therefore, in this implementation, the user's mobile device can: receive selection of a first visualization model for visualizing emotions from the user; generate a first visualization of a timeseries of emotions in an emotion event according to the first visualization model; and locally render this first visualization for the user. Responsive to the user electing a recipient of this emotion file, the user's mobile device (or the remote computer system) can also upload the emotion file to a remote database for storage and authorize access to the emotion file by the recipient. Later, a second computing device affiliated with the recipient can: access the emotion file from the database; receive selection of a second visualization model for visualizing the emotion file from the second user; generate a second visualization of a timeseries of emotions—represented in the emotion file—according to the second visualization model; and render this second visualization on an integrated or connected display.

### 9.2 Other Representations

The user's mobile device or another computing device can implement similar methods and techniques to output representations of the user's experience when replaying the emotion file in order sensory domains, such as audible, tactile, or olfactory representations of emotions recorded in the emotion file.

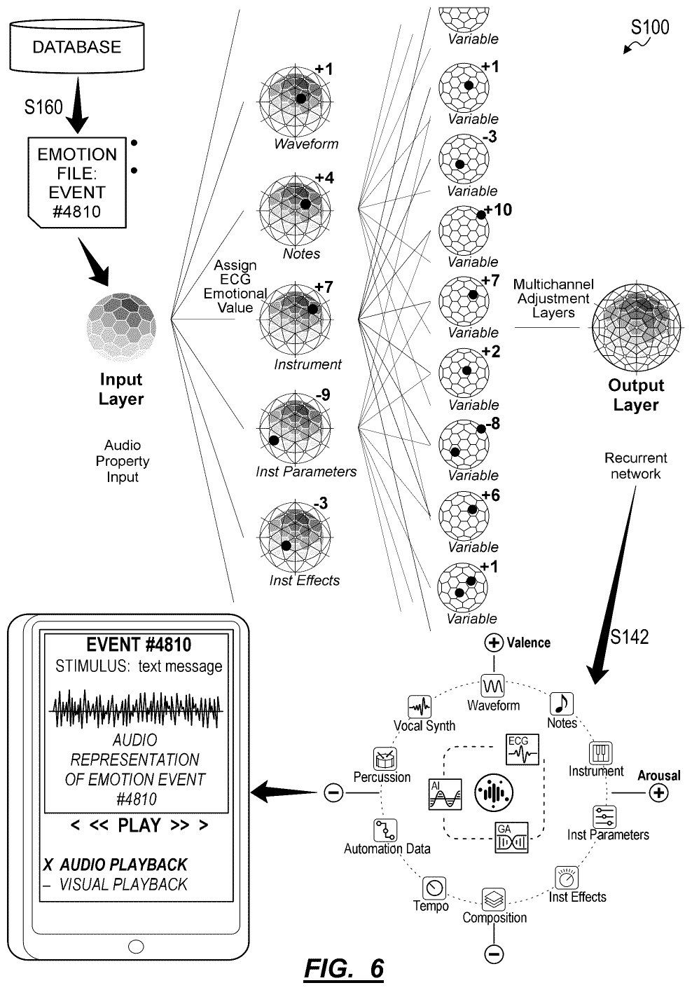

In one example shown in FIG. 6, the mobile device (or the remote computer system) transforms the emotion file into an audible representation of the user's emotions during the emotion event, such as by: linking a particular synthetic instrument to a biosignal or emotion type; varying synthetic outputs of a set of synthetic instruments according to intensities of corresponding emotions or biosignals detected in the emotion file over the duration of the emotion file to form a synthetic ensemble; and then setting or varying a tempo of this synthetic ensemble according to intensities of emotions or biosignals represented in this emotion file. The mobile device can then replay this synthetic ensemble for the user or send this synthetic ensemble to a recipient selected by the user.

### 10. Replayed Experience

In one variation, the system can execute the foregoing methods and techniques to generate and store an emotion file that can be replayed at a later time in order to enable others (e.g., a therapist, a guardian, a family member) to gain insight into the user's emotional experience during an emotion event.

### 10.1 Example: Gift Response

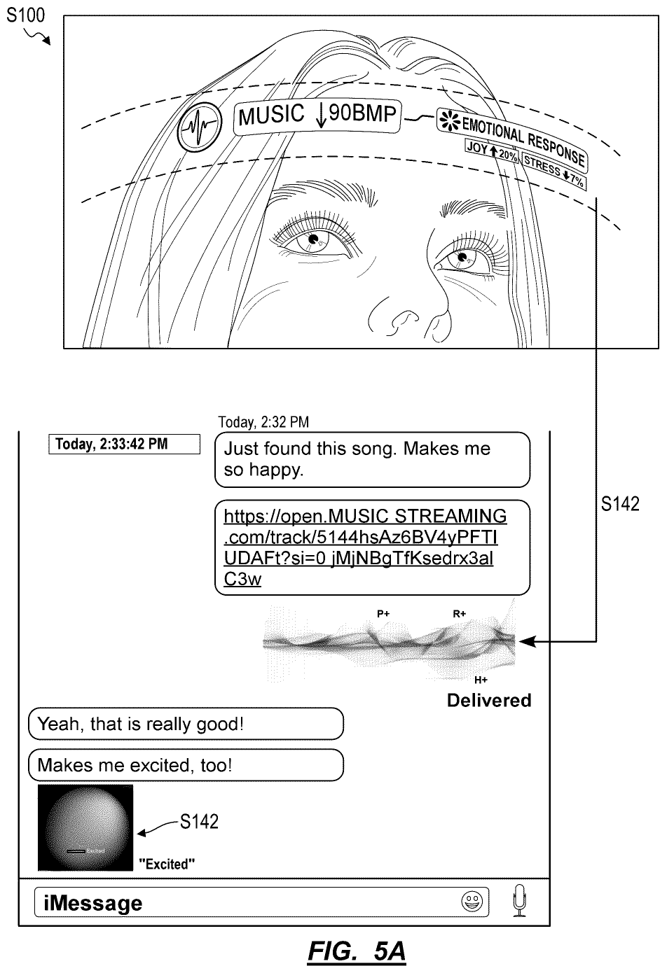

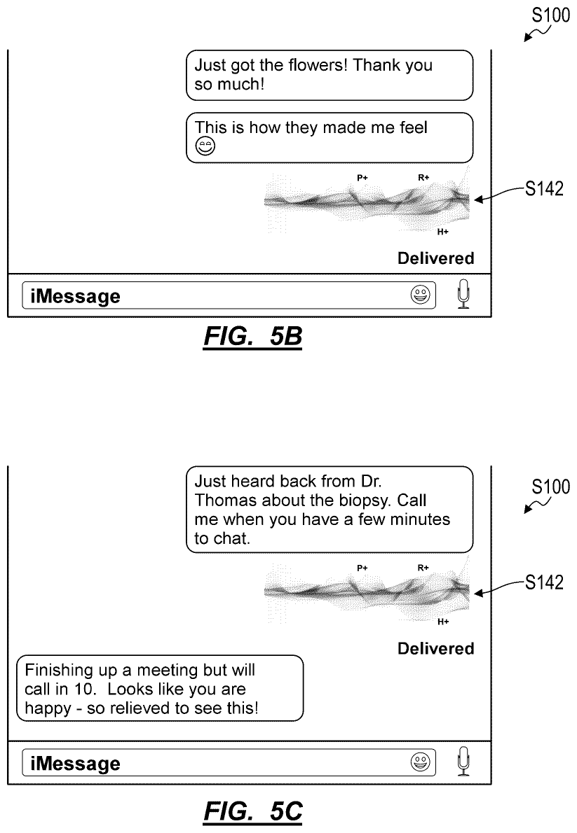

In one example shown in FIG. 5B, the user manually triggers an emotion event at her wearable device or mobile device after receiving a gift from a sender in order to capture her emotions during receipt of this gift and then returns the resulting emotion file to the sender in order to share her emotional response to the gift with this sender.

In this example, the user may: receive the gift from the sender; open the gift; experience a positive emotional response to receiving the gift; elect to share this emotional response with the sender; and tap a button on the wearable device accordingly in order to trigger the wearable device to retrieve contents from the rolling buffer and initiate generation of an emotion file from these raw biosignal data. The wearable device can then offload these raw biosignal data to the user's mobile device, and the user's mobile device (or the remote computer system) can then transform these raw biosignal data into an emotion file and replay a visualization of this emotion file, as described above. The mobile device can also prompt the user: to trim the emotion file to span a relevant time period around receipt of the gift; to annotate the emotion file; and to select a recipient (i.e., the sender who sent the gift) of the emotion file from a contacts list. The mobile device can then send the clipped and annotated emotion file (or the final clipped and annotated visualization) to the recipient, such as in the form of a MMS text message or notification within a native application. The recipient's computing device can then play back the emotion file in a visualization, thereby sharing with the recipient—who sent the gift to the user—how the user experienced receiving the gift, which may be more authentic and coherent than merely a “thank you” from the user.

In a similar example, while a user is present in an office, a delivery person enters the office with flowers. The user notices these flowers and considers the intended recipient. When the delivery person delivers these flowers to the user, the user experiences both surprise and excitement. Upon finding a card with the flowers and reading the name of the sender, the user experiences happiness and then considers sharing this emotional experience with the sender, including: interest and curiosity upon seeing these flowers enter the office; then surprise and excitement upon realizing the flowers are for her; and then happiness upon learning the identity of the sender. Accordingly, the user triggers the wearable device to record biosignal data stored in the local buffer—captured by the wearable device automatically during this emotion event—and to initiate generation of an emotion file for this emotion event.

In this example, upon receipt of these raw biosignal data from the wearable device and interpretation of a timeseries of emotions from these raw biosignal data, the mobile device can: scan this emotion timeseries for changes in type and magnitude of detected emotions (e.g., steady state, then a shift to interest and curiosity, then a shift to surprise and excitement, followed by a shift to happiness); place keypoints in the emotion file at these changes in type and magnitude of detected emotions; and prompt the user to manually annotated these keypoints. For example, the user may label these keypoints as: “saw the flowers”; “realized the flowers were for me”; “saw your name on the card”; and “thank you!” The mobile device can transmit this annotated emotion file to a second user (i.e., the sender) selected by the user, such as in the form of a MMS message or a notification within a native application. Upon receipt of this annotated emotion file, the second user's computing device can generate and play back a visualization of the emotion file—including synchronized keypoint labels entered by the user—thereby enabling the second user to visualize the user's emotional response to receiving these flowers from the second user and thus enabling the second user to gain greater insight into the significance of the gift for the user than a mere “thank you.”

### 10.2 Example: Music Playback

In a similar example, the system can: generate an emotion file for a user while the user listens to a song title; synchronize a digital copy of the song title with the emotion file to form a private multimedia song file; and store the private multimedia song file in a local or remote database. The user may then share the private multimedia song file with a friend or family member, who may then playback the private multimedia song file at his own mobile device in order perceive emotions experienced by the user while listening to this this song title.

In a similar example shown in FIG. 5A, the user listens to music via a music streaming application executing on her mobile device while wearing the wearable device. Upon hearing a particular song title, the user experiences a positive emotion, considers sending this song title and a visualization of this positive emotion to a friend, and then selects a “share” option within the music streaming application. Accordingly, the music streaming application can prompt the user to elect to share this song title with an emotion file. If confirmed by the user, the mobile device can: transmit a query to the wearable device for contents of the rolling buffer; transform raw biosignal data received from the wearable device into an emotion file; retrieve a start time and end time of playback of the song title at the mobile device; and automatically trim the emotion file to this start time and end time. The music streaming application can also prompt the user to select a recipient from a contact list and playback a visualization of the emotion file before prompting the user to confirm transmission of the emotion file and a link to the song title to the recipient. Once confirmed by the user, the mobile device can transmit a link to the song title and the emotion file (or a link to the emotion file) to the recipient's computing device.

Once the recipient selects this song title link at her computing device, her computing device can: playback the song title; and render a visualization of the emotion file synchronized to the song title. (In this example, the music streaming application can also: generate a static visualization of emotions represented in the emotion file, such as a static graphical image depicting a range of emotions detected in biosignal data captured as the user listened to the song title; write a link for the song title to this static visualization; and transmit this static visualization to the recipient. When the recipient selects this static visualization, the recipient's computing device can playback the song title and concurrently render a synchronized animated visualization of the emotion file.)

In this example, the recipient's computing device can implement similar methods and techniques: to generate a second emotion file and/or a second visualization representing the recipient's emotional response to listening to the song title; to render the second visualization for the recipient; and to then return this second emotion file and/or a second visualization back to the user, as shown in FIG. 5A, thereby enabling the user and the recipient to visualize and compare their emotional responses to the same song title.

Therefore, in this example, the user's wearable device and mobile device can cooperate to: present a media (e.g., playback a song title) to the user during a period of time; receive selection of a recipient—for the media—from an electronic contact list; prompt the user to pair the media with personal emotion status data (e.g., an emotion file for the period of time); retrieve biosignal data from the rolling buffer on the wearable device in response to receiving confirmation from the user to pair the media with personal emotion status data; transform these biosignal data into a timeseries of emotions exhibited by the user while consuming the media; and then transmit the media (or a link to the media) and the emotion file (or the visualization of the emotion file) to the recipient's computing device. The recipient's computing device can then render a visualization of the user's emotion file synchronized to playback of the media.

The user's wearable device and mobile device (and/or the remote computer system) can implement similar methods and techniques: to generate an emotion interpretation model file representing emotions exhibited by the user while consuming a film, a video clip, an audio broadcast, a book, a news article, a blog post, a multimedia message, or other media; and to pair or link this emotion file to this media.

### 10.3 Example: Receiving Message

In another example, the user manually triggers an emotion event at her wearable device or mobile device after receiving a message from a sender in order to capture her emotions during review of this message and then returns the resulting emotion file to the sender in order to share her emotional response to the message with this sender.

In this example, when the user receives and reads a message (e.g., SMS or MMS text message, an email) from a sender within a native messaging application executing on her mobile device, the user may experience an emotional response to the message, elect to share this emotional response with the sender, and manually select a virtual emotion capture button within this native messaging application. Responsive to selection of this virtual emotion capture button, the mobile device can: query the wearable device for emotion data from the rolling buffer; store a selection time of the virtual emotion capture button; prompt the user to select an emotion capture duration (e.g., five, ten, 30, or 60 seconds from a dropdown menu); and trim biosignal data received from the wearable device to a period spanning emotion capture duration and terminating at the selection time. The mobile device can then: implement methods and techniques described above to transform these trimmed biosignal data into an emotion file; (automatically annotate this emotion file with an identify of the sender, content from the message, and an external stimulus trigger for consumption of a message); and then send the emotion file to the sender via the native messaging application once confirmed by the user. Upon receipt of the emotion file, the sender's computing device can generate and render a visualization of this emotion file, thereby enabling the sender to gain better insight into the user's visceral emotional response to the sender's previous message. More specifically, the mobile device can thus enable the user to show the sender how this message effected the user's emotions rather than merely describe this effect in words.

Therefore, in this example, the mobile device can: retrieve biosignal data from the rolling buffer in the wearable device in response to receipt of a manual trigger from the user following receipt of a media (e.g., a text message) from a sender; transform these biosignal data into a timeseries of emotions exhibited by the user while consuming the media; generate a visualization (and/or an emotion file) depicting a change in emotional state of the user during consumption of the media; and transmit the visualization (or the emotion file) to a computing device associated with the sender.

### 10.4 Example: Sending Message

In a similar example shown in FIG. 5C, the user manually triggers an emotion event at her wearable device or mobile device while or after drafting a message to a recipient in order to capture her emotions during composition of this message and then returns the resulting emotion file to the sender in order to better articulate her intent in this message to the recipient. More specifically, in this example, the user's wearable mobile device (and/or the remote computer system) can execute Blocks of the method S100 to pair a message composed by the user with an emotion file (or a visualization) representing the user's emotions while composing this message and then send this message—with the emotion file (or the visualization)—to a recipient selected by the user, thereby: enabling the recipient to access the user's emotional state while composing the message and to interpret this message in the context of the user's emotion state; and reducing opportunity for the recipient to misconstrue the user's intent in the message.

For example, after drafting a message—designating a recipient—within a native messaging application executing on the mobile device, the user selects a virtual emotion capture button within the native messaging application to trigger the mobile device to pair this message with an emotion file (or a visualization) representing her emotional state while composing this message. In particular, user may be concerned that the intent of her message may be ambiguous or may be misinterpreted and may therefore elect to pair this message with a representation of her emotional state while composing the message in order to reduce ambiguity and opportunity of the recipient to misinterpret this message. Accordingly, the mobile device can implement methods and techniques described above: to retrieve biosignal data from the rolling buffer; to trim this biosignal data; to transform these trimmed biosignal data into an emotion file (and/or a visualization); to pair this emotion file (and/or the visualization) with the user's message; and to send the augmented message to the recipient. The recipient's computing device can then render the user's message and a visualization of the user's emotion while composing this message for the recipient, thereby enabling the recipient to better comprehend the user's emotional state while drafting the message and thus more accurately interpret the intended meaning of this message.

In the foregoing example, the user may elect to pair a message with a visualization of her emotional state before drafting the message, and the user's mobile device can implement similar methods and techniques to automatically: retrieve live emotion data from the wearable device; and assemble these emotion data into an emotion file and/or a visualization in (near) real-time as the user composes the message.

Additionally or alternatively, if the user send a first message to the recipient and then receives a second message—back from the recipient—that suggests a misunderstanding of the intent of the first message, the user may elect to return an emotion file representing the user's emotional state while composing the first message back to the recipient. Accordingly, the mobile device can retrieve contents of the rolling buffer from the wearable device and automatically trim these biosignal data to a period of time over which the user previously composed the first message. For example, the mobile device can trim these biosignal data to a period of time preceding a transmission timestamp of the first message (e.g., to a period of 30 seconds preceding this transmission timestamp) or prompt the user to manually trim these biosignal data. The mobile device can then; generate an emotion file for this period of time back to the recipient; prompt the user to enter a third message (e.g., “That isn't what I meant, and no, I am not upset. Here is my emotional state when I sent you that message.”); and then return this emotion file and the third message to the recipient, thereby enabling the recipient to better understand the user's intent of the first message.

Therefore, in this example, the user's wearable device and mobile device (and/or the remote computer system) can: receive selection of a recipient from an electronic contact list; record a message entered by the user over a period of time; prompt the user to pair the message with personal emotion status data; retrieve biosignal data from the rolling buffer in response to receiving confirmation from the user to pair the message with personal emotion status data; transform the set of biosignal data into the timeseries of emotions exhibited by the user during entry of the message; and then transmit the message and an emotion file (or a visualization) to a computing device affiliated with the recipient. The recipient's computing device can then render a visualization of this emotion file with the message for the recipient.

### 10.5 Example: Music Creation

In another implementation, a song title is synchronized and stored with an emotion file—generated from biosignal data of the artist generated during recordation of the song title—to form a multimedia song file. When the multimedia song file is replayed on a user's mobile device, the mobile device can: output an audio track; and render a visualization—such as described above—of timeseries emotion data (e.g., a virtual avatar or virtual wax ball with a heart rate counter) synchronized to this audio track, thereby enabling the user to better perceive emotions elicited in the artist when singing or playing the song title.

In one example, the user wears the wearable device while recording a song and selects a button on the wearable device to trigger generation of an emotion file during the song (e.g., after experiencing a meaningful response from audience members) or after the user completes the song (e.g., after a successful take in a recording studio). Then, in response to receiving this manual trigger from the user during this performance, the wearable device, the mobile device, and/or the remote computer system can implement methods and techniques described above to: retrieve biosignal data from the rolling buffer; derive a timeseries of emotions from these biosignal data; write this timeseries of emotions to an emotion file; and link this emotion file to a recording of the performance. For example, the mobile device or the remote computer system can: trim and align the emotion file to a duration of the song; synchronize emotions represented in the emotion file to a recording of the song; and store the emotion file and the recording of the song in one multimedia file or otherwise define a link between the emotion file and the recording of the song. The recording of the song and the emotion file can then be published to a media service, such as a music streaming platform.

Later, in response to selection of the song by a second user at a second computing device, the music streaming platform can retrieve the emotion file—linked to this song—from the remote database and serve the emotion file with the recording of the song to the second computing device. The second computing device can then: implement methods and techniques described above to generate a visualization of the emotion file; playback the recording of the song; and render the visualization—synchronized to playback of the recording of the song—on a display of the second computing device.

The user's mobile device can implement similar methods and techniques to generate an emotion file based on biosignal data captured by the wearable device as the user performs a comedy sketch, performs an act in a play, competes in a sporting event, or performs any other action. The wearable device, the mobile device, and/or the remote computer system can then implement similar methods and techniques to generate and synchronize an emotion file to an audio recording or video recording of this action by the user and to link this emotion file to the audio or video recording. Thus, when another user views this audio or video recording (e.g., on an audio or video streaming platform), a consumer consuming this audio or video recording may also gain insight into the user's emotional experience during this action via a visualization of this emotion file generated from user biosignal data captured during this action.

### 10.6 Example: Wedding

In another example, a user manually triggers recordation of biosignal data and generation of an emotion file during the user's wedding ceremony. The system also accesses a timestamped video recording from the wedding ceremony, and synchronizes the video recording and emotion file of the user (and an emotion file of the user's spouse) to generate a multimedia video of the wedding ceremony. Thus, during playback of the multimedia video, at an emotion file viewer, the emotion file viewer can render the video, output an audio track of the ceremony, and render an emotion graph or virtual avatar depicting the user's emotions (and the spouse's emotions) during the wedding ceremony.

The system can implement similar methods and techniques to generate multimedia videos of other life events or milestones, such as birth of a child, a graduation, a sporting event, or an award ceremony. The system can also save these multimedia videos in a remote database or locally on a user's mobile device, and the user may share these multimedia videos with family, friends, etc.—over time and over distances—in order to provide greater insight into her emotions during these events.

### 10.7 Example: Therapy

In another example, the user manually indicates an emotion event when experiencing a positive or negative emotion type of interest, thereby triggering the system to generate an emotion file representative of the user's emotions during this emotion event. The user may then share this emotion file with her therapist, such as to enable the therapist to better comprehend and address the user's response to this experience.

### 10.8 Example: Non-Verbal User

In a similar example, a non-verbal child manually indicates an emotion event when experiencing a positive or negative emotion type of interest, such as when bullied at school or when being recognized for something positive in class, thereby triggering the system to generate an emotion file representative of the user's emotions during this emotion event. In this example, the child may later share this emotion file with her parent in order communicate to the parent how this earlier experience at school made her feel.

In this example, the system can generate the emotion file in (near) real-time, and the child's mobile device can render a visualization in the emotion in (near) real-time, thereby enabling the child to better communicate her feelings to others via her mobile device.

### 11. Real-time Emotion Capture

In another variation, the system can execute the foregoing methods and techniques to generate an emotion file that can be replayed in (near) real-time as a visualization of the user's emotions in order to enable others (e.g., a therapist, a guardian, a family member, a music platform, a media platform) to gain insight into the user's current emotional experience and respond accordingly in (near) real-time.

Similarly, for two users wearing similar biosignal-enabled wearable devices (e.g., EEG-enabled earbuds or headphones) connected via a computer network, the remote computer system can: collect biosignal data from these wearable devices; interpret emotions of the two users in real-time; and distributed representations emotions of each user to the opposing user in visual, audible, haptic, and/or olfactory sensory domains.

### 11.1 Recorded Music

In another example, the system automatically detects and tracks the user's emotions and emotion intensities while listening to music at her mobile device, renders a visualization of these detected emotions as described in (near) real-time, and generates an emotion file depicting the user's emotions tagged with song titles played back by the mobile device during this period.

The system can also: automatically track emotion changes and concurrent music features (e.g., tempo, amplitude, genre, timbre) during this period of time; derive correlations between user emotion changes, user emotion intensities, and these music features, such as a function of people nearby, locations, time of day, etc.; and store these correlations in an emotion interpretation model unique to the user. Later (or in near real-time), the system can select from a corpus of existing songs or generate custom audio streams to achieve a target emotion or emotion intensity in the user based on this emotion interpretation model.

### 11.2 Example: Live Music

In another example, an artist wears her wearable device during a live performance, and the system implements methods and techniques described above to generate an emotion file based on biosignal data recorded by the wearable device during this live performance. The system can also: interface with a large display in the concert hall to render a live visualization of the artist's emotions during the live performance and/or store the emotion file for later access by fans, such as with a video or audio recording of the performance. The system can thereby visualize the artist's emotions during the live performance in order to communicate greater authenticity of the artist to fans in the concert hall.

### 12. Searchable Emotion Events

As described above, the mobile device (and/or the remote computer system) can upload an emotion file for an emotion to a remote database for storage and later retrieval.

In particular, the mobile device (and/or the remote computer system) can cooperate with the user to annotate or tag an emotion file with: an identity of the user; internal and/or external stimuli that trigger the emotion event; actions by the user leading up to and/or during the emotion event; a date, time, and/or duration of the emotion event; a link to media generated by the user during the emotion event; and/or other metadata. The mobile device (and/or the remote computer system) can then store this emotion file in a searchable database. Later, the user may log in to an emotion-tracking portal—such as within a native application or web browser executing on her mobile device—and enter a set of search terms. For example, the emotion-tracking portal can prompt or enable the user to search and filter through stored emotion files of past emotion events based on: date range; geospatial location; primary emotion detected; transition from a first emotion to a second emotion; change in emotion magnitude (or “intensity”); rate of emotion type or emotion magnitude change; stimulus type (i.e., internal or external); stimulus keyword; emotion event duration; and/or user action; etc.

The remote database can then return a set of emotion files containing tags and other metadata that match or approximate these search terms to the emotion-tracking portal. When the user selects a particular emotion file from this set, the mobile device can: download a local copy of this emotion file; generate a visualization of a past emotion event represented in this emotion file (e.g., based on a type of visualization selected by the user); and then playback this visualization for the user, such as with static or animated textual descriptions (e.g., of stimuli) at emotion keypoints along the duration of the emotion event, thereby enabling the user to review (or “look back on”) this past emotion event. (Alternatively, the remote controller can generate this visualization remotely and return this visualization to the user's mobile device for local playback.)

### 12.1 Stored Raw Data File

In this variation, the user's wearable device, the user's mobile device, and/or the remote computer system can implement similar methods and techniques: to annotate or tag a raw data file for an emotion event with metadata; to store this raw data file in the remote database; and to retrieve this raw data file when selected by the user with the emotion-tracking portal. The user's mobile device (or the remote computer system) can then implement a current emotion interpretation model—such as a current generic emotion interpretation model for a population of users or a custom emotion interpretation model generated uniquely for the user based on past emotion data and feedback from the user—to interpret a timeseries of emotions from this raw data file. In particular, because the system can improve an emotion interpretation model for interpreting emotions from raw biosignal data over time as the system collects more emotion data and feedback from the user and/or from a population of users, the system can store a raw emotion file for an emotion event and then implement a current instance of the emotion interpretation model to interpret a timeseries of emotions from this raw data file when this raw data file is called by the user, thereby enabling the system (e.g., the mobile device and/or the remote computer system) to improve emotion type and magnitude interpretation for this emotion event over time.

The user's mobile device (and/or the remote computer system) can then: generate a visualization of the emotion event represented in this timeseries of emotion extracted from this raw data file; and playback this visualization for the user, thereby enabling the user to review this past emotion event in light of current (e.g., refined, customized) emotion detection and interpretation methods.

Therefore, in this variation, the user's wearable device and mobile device (and/or the remote computer system) can cooperate: to write biosignal data—spanning an emotion event during a first time—to a raw data file; to store the raw data file in a remote database; to transform these biosignal data into a first timeseries of emotions based on a first stored emotion interpretation model for interpreting emotions from raw biosignal data available at the first time (e.g., a generic emotion interpretation model); and to generate and render a first visualization of this first timeseries of emotions for the user. When the user (or other entity authorized to access this raw data file) selects this raw data file at a later time (e.g., hours, data, weeks, or years after the emotion event), the remote computer system and/or the user's mobile device can: retrieve the raw data file from the remote database; access a second emotion interpretation model (such as customized for the user based on new emotion and feedback data collected from the user since the emotion event or refined for a population of users based on new emotion and feedback data collected from the population of users since the emotion event); transform the set of biosignal data—stored in the raw data file—into a revised timeseries of emotions based on this second emotion interpretation model for interpreting emotions from raw biosignal data available at this later time; generate a second visualization of this revised timeseries of emotions; and then render this second visualization of the revised timeseries of emotions for the user.

Therefore, by storing a raw data file rather than (only) an emotion file or (only) a visualization of an emotion event, the system can enable improved interpretation of types and magnitudes of emotion exhibited by the user during this emotion event over time as the system improves an emotion interpretation model for the user or for a population of users more generally over time.

### 12.2 Refined Emotion and Visualization Models

Similarly, the system can publish new and/or refined visualization models—for visualizing timeseries of emotions interpreted from raw biosignal data collected during an emotion event—over time. When the user (or other entity authorized to access this raw data file) selects an emotion file or a raw data file—representing an earlier emotion event—from a database at an emotion-tracking portal, the emotion-tracking portal can prompt the user to select from a list of current visualization models and generate and render a visualization of this emotion event based on the selected visualization model.

Therefore, by storing a raw data file and/or an emotion file for an emotion event rather than (only) a visualization of the emotion event, the system can enable the user and/or other entities authorized to view this emotion event to view this emotion event in their preferred visualization formats and with visualization formats that are improved and refined over time.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

1. A method for deriving and storing emotional conditions of humans includes:
during a global time period, writing timeseries biosignal data, output by a set of biosensors in a local device worn by a user, to a rolling buffer spanning a fixed look-back duration;
in response to a trigger event at a first time during the global time period:
retrieving a first set of biosignal data, recorded during the global time period prior to the first time, from the rolling buffer;
recording a second set of biosignal data following the trigger event at the first time;
writing a set of biosignal data to a raw data file, the set of biosignal data comprising the first set of biosignal data and the second set of biosignal data recorded continuously during a first time period within the global time period and spanning the trigger event at the first time, to a raw data file; and
storing the raw data file in a database;
transforming the first set of biosignal data into a first timeseries of emotions exhibited by the user during the first time period;
associating the first timeseries of emotions with the trigger event;
generating a visualization of the first timeseries of emotions, the visualization representing emotions exhibited by the user during the first time period;
in response to receiving a first query for a characteristic of the trigger event, triggering playback of the visualization of the first timeseries of emotions on a display of a computing device; and
in response to receiving a second query for a particular emotion in the first timeseries of emotions, triggering playback of the visualization of the first timeseries of emotions on the display of the computing device.

2. The method of claim 1:
further comprising:
detecting a change in emotional state of the user proximal the first time based on timeseries biosignal data output by the set of biosensors and written to the rolling buffer in the local device; and
interpreting the trigger event based on the change in emotional state of the user; and
wherein rendering the visualization of the first timeseries of emotions on the display comprises rendering the visualization of the first timeseries of emotions on the display in communication with the local device.

3. The method of claim 2, further comprising:
generating a notification comprising a prompt to indicate an external stimulus that initiated the change in emotional state of the user;
transmitting the notification to the user;
in response to receiving identification of the external stimulus from the user:
labeling the raw data file with the external stimulus identified by the user; and
storing the raw data file in a database;
at a second time, succeeding the first time, receiving a search term from the user; and
in response to the search term matching the external stimulus:
retrieving the raw data file;
regenerating the visualization of the first timeseries of emotions; and
rendering the visualization for the user.

4. The method of claim 2:
wherein retrieving the first set of biosignal data from the rolling buffer in response to detecting the trigger event comprises, in response to detecting the change in emotional state of the user:
retrieving the set of biosignal data, representing the change in emotional state, from the rolling buffer; and
writing the set of biosignal data, spanning the first time period preceding the first time, to a raw data file;
further comprising:
in response to detecting the change in emotional state of the user, writing a second set of biosignal data, output by the set of biosensors in the local device over a second time period succeeding the first time, to the raw data file; and
transforming the second set of biosignal data in the raw data file into a second timeseries of emotions exhibited by the user during the second time period; and
wherein generating the visualization comprises generating the visualization of the first timeseries of emotions and the second timeseries of emotions.

5. The method of claim 1:
further comprising, during the global time period, discarding timeseries biosignal data, from the rolling buffer, output by the set of biosensors during time periods preceding the fixed look-back duration; and
wherein retrieving the first set of biosignal data, spanning the first time period and preceding the first time, from the rolling buffer comprises retrieving the first set of biosignal data from the rolling buffer, the first set of biosignal data spanning the first time period, preceding the first time, and output by the set of biosensors within the fixed look-back duration of the first time.

6. The method of claim 1:
wherein transforming the first set of biosignal data into the first timeseries of emotions exhibited by the user during the first time period comprises transforming the first set of biosignal data into the first timeseries of emotions exhibited by the user during the first time period based on a first emotion detection model available during a second time period;
wherein triggering playback of the visualization of the first timeseries of emotions on the display of the computing device in response to receiving the first query for the trigger event comprises triggering playback of the visualization of the first timeseries of emotions on the display of the computing device during the second time period in response to receiving the first query for the trigger event during the second time period;
further comprising:
retransforming the first set of biosignal data into the first timeseries of emotions exhibited by the user during the first time period based on a second emotion detection model available during a third time period succeeding the second time period; and
generating the visualization of the first timeseries of emotions during the third time period; and
wherein triggering playback of the visualization of the first timeseries of emotions on the display of the computing device in response to receiving the second query for the particular emotion in the first timeseries of emotions comprises triggering playback of the visualization of the first timeseries of emotions on the display of the computing device during the third time period in response to receiving the second query for the particular emotion in the first timeseries of emotions during the third time period.

7. The method of claim 1:
wherein transforming the first set of biosignal data into the first timeseries of emotions comprises transforming the first set of biosignal data into the first timeseries of emotions based on a first model for interpreting emotions from raw biosignal data available at the first time; and
further comprising, at a second time succeeding the first time:
retrieving the raw data file from the database;
transforming the first set of biosignal data, stored in the raw data file, into a revised timeseries of emotions based on a second model for interpreting emotions from raw biosignal data available at the second time;
generating a second visualization of the revised timeseries of emotions;
in response to receiving a third query for the trigger event, triggering playback of the second visualization of the revised timeseries of emotions on the display of the computing device; and
in response to receiving a fourth query for a second particular emotion in the first timeseries of emotions, triggering playback of the second visualization of the revised timeseries of emotions on the display of the computing device.

8. The method of claim 1:
further comprising interpreting a change in emotional state of the user, from a first emotion type to an emotion type of interest preselected by the user, proximal the first time based on timeseries biosignal data output by the set of biosensors in the local device;
wherein retrieving the first set of biosignal data from the rolling buffer in response to the trigger event comprises, in response to detecting the change in emotional state of the user to the emotion type of interest:
retrieving the first set of biosignal data, representing the change in emotional state, from the rolling buffer; and
writing the first set of biosignal data, spanning the first time period preceding the first time, to a raw data file; and
further comprising:
prompting the user to indicate an external stimulus that initiated the change in emotional state of the user;
labeling the raw data file with the external stimulus identified by the user; and
storing the raw data file in a database.

9. The method of claim 1, further comprising:
at a second time succeeding the first time, receiving a set of search terms from the user; and
in response to the set of search terms matching the emotion type of interest and the external stimulus:
retrieving the raw data file;
regenerating the visualization of the first timeseries of emotions; and
in response to receiving a third query for the trigger event, triggering playback of the visualization on the display; and
in response to receiving a fourth query for a second particular emotion in the first timeseries of emotions, triggering playback of the visualization on the display.

10. The method of claim 1:
wherein writing timeseries biosignal data output by the set of biosensors to the rolling buffer comprises writing timeseries biosignal data output by the set of biosensors to the rolling buffer defining the fixed look-back duration of approximately five minutes, the set of biosensors comprising a heart rate sensor, a skin temperature sensor, and a galvanic skin response sensor integrated into the local device worn by the user;
wherein retrieving the first set of biosignal data from the rolling buffer comprises, at a mobile computing device affiliated with the local device:
querying the local device for contents of the rolling buffer in response to manual selection of a capture trigger at the local device; and
downloading the first set of biosignal data from the rolling buffer; and
wherein triggering playback of the visualization of the first timeseries of emotions on the display comprises triggering playback of the visualization of the first timeseries of emotions on the display of the mobile computing device.

11. The method of claim 1, wherein generating the visualization of the first timeseries of emotions and rendering the visualization of the first timeseries of emotions on the display comprises, at the computing device, during a second time period succeeding the first time period, during playback of the visualization on the display:
rendering an emotion graph depicting a set of emotion axes;
at a first playback time, rendering an avatar at a first position on the emotion graph, the first position corresponding to a first type and a first magnitude of a first emotion in the first timeseries of emotions exhibited by the user during the first time period;
at a second playback time succeeding the first playback time, transitioning the avatar to a second position on the emotion graph, the second position corresponding to a second type and a second magnitude of a second emotion in the first timeseries of emotions exhibited by the user during the first time period; and
at a third playback time succeeding the second playback time, transitioning the avatar to a third position on the emotion graph, the third position corresponding to the second type and a third magnitude of the second emotion in the first timeseries of emotions exhibited by the user during the first time period.

12. The method of claim 1:
further comprising:
receiving selection of a recipient from an electronic contact list;
recording a message entered by the user over the first time period; and
prompting the user to pair the message with personal emotion status data;
wherein retrieving the first set of biosignal data from the rolling buffer comprises retrieving the first set of biosignal data from the rolling buffer in response to receiving confirmation from the user to pair the message with personal emotion status data;
wherein transforming the first set of biosignal data into the first timeseries of emotions comprises transforming the first set of biosignal data into the first timeseries of emotions exhibited by the user during entry of the message;
further comprising, in response to receiving confirmation from the user to pair the message with personal emotion status data, transmitting the message and the visualization to a second computing device affiliated with the recipient; and
wherein rendering the visualization of the first timeseries of emotions on the display comprises rendering the visualization with the message on the display of the second computing device.

13. A method for deriving and storing emotional conditions of humans comprising:
at a local device worn by a user:
writing timeseries biosignal data, output by a set of biosensors in the local device, to a rolling buffer spanning a fixed look-back duration;
discarding timeseries biosignal data, from the rolling buffer, output by the set of biosensors during an initial time period preceding the fixed look-back duration; and
in response to a trigger event at a first time:
writing timeseries biosignal data, contained in the rolling buffer and spanning a first time period preceding the first time and within the fixed look-back duration, to a raw data file associated with the trigger event; and
writing timeseries biosignal data, spanning a second time period succeeding the first time, to the raw data file;
transforming timeseries biosignal data in the raw data file into a first timeseries of emotions exhibited by the user;
associating the first timeseries of emotions with the trigger event;
generating a visualization of the first timeseries of emotions; and
rendering the visualization of the first timeseries of emotions on a display of a computing device.

14. The method of claim 13, wherein rendering the visualization of the first timeseries of emotions on the display of the computing device comprises, in response to receiving a first query for the trigger event, triggering playback of the visualization of the first timeseries of emotions on the display of the computing device.

15. The method of claim 13, wherein rendering the visualization of the first timeseries of emotions on the display of the computing device comprises:
in response to receiving a first query for a characteristic of the trigger event, rendering the visualization of the first timeseries of emotions on the display of the computing device; and
in response to receiving a second query for a particular emotion in the first timeseries of emotions, rendering the visualization of the first timeseries of emotions on the display of the computing device.

16. The method of claim 15:
wherein transforming timeseries biosignal data into the first timeseries of emotions exhibited by the user comprises transforming timeseries biosignal data into the first timeseries of emotions exhibited by the user during the first time period based on a first emotion detection model available during a second time period;
wherein triggering playback of the visualization of the first timeseries of emotions on the display of the computing device in response to receiving the first query for the trigger event comprises triggering playback of the visualization of the first timeseries of emotions on the display of the computing device during the second time period in response to receiving the first query for the trigger event during the second time period;
further comprising:
retransforming timeseries biosignal data in the raw data file into the first timeseries of emotions exhibited by the user during the first time period based on a second emotion detection model available during a third time period succeeding the second time period; and
generating the visualization of the first timeseries of emotions during the third time period; and
wherein triggering playback of the visualization of the first timeseries of emotions on the display of the computing device in response to receiving the second query for the particular emotion in the first timeseries of emotions comprises triggering playback of the visualization of the first timeseries of emotions on the display of the computing device during the third time period in response to receiving the second query for the particular emotion in the first timeseries of emotions during the third time period.

17. The method of claim 13:
further comprising:
detecting a change in emotional state of the user proximal the first time based on timeseries biosignal data output by the set of biosensors and written to the rolling buffer in the local device; and
interpreting the trigger event based on the change in emotional state of the user; and
wherein rendering the visualization of the first timeseries of emotions on the display comprises rendering the visualization of the first timeseries of emotions on the display in communication with the local device.

18. A method for deriving and storing emotional conditions of humans comprising:
writing timeseries biosignal data, output by a set of biosensors in a local device coupled to a user, to a rolling buffer spanning a fixed look-back duration;
interpreting a change in emotional state of the user proximal a first time based on timeseries biosignal data output by the set of biosensors in the local device;
in response to interpreting the change in emotional state proximal the first time, retrieving a first set of biosignal data, spanning a first time period preceding the first time, from the rolling buffer;
transforming the first set of biosignal data into a timeseries of emotions exhibited by the user during the first time period;
associating the timeseries of emotions with the change in emotional state;
generating a notification comprising a prompt to indicate an external stimulus that initiated the change in emotional state of the user;
transmitting the prompt to the user;
in response to receiving identification of the external stimulus from the user:
labelling the first timeseries of emotions within the external stimulus identified by the user; and
storing the first timeseries of emotions, labeled with the external stimulus, in an emotion file associated with the user;
storing the emotion file in a database;
in response to receiving selection of the emotion file from the user, generating a visualization of the external stimulus and the first timeseries of emotions stored in the emotion file; and
in response to receiving a first query for the first timeseries of emotions, rendering the visualization of the first timeseries of emotions on a display of a computing device.

19. The method of claim 18:
further comprising:
detecting a change in emotional state of the user proximal the first time based on timeseries biosignal data output by the set of biosensors and written to the rolling buffer in the local device; and
interpreting the trigger event based on the change in emotional state of the user;
wherein rendering the visualization of the first timeseries of emotions on the display comprises rendering the visualization of the first timeseries of emotions on the display in communication with the local device;
further comprising:
generating a notification comprising a prompt to indicate an external stimulus that initiated the change in emotional state of the user;
transmitting the notification to the user;
in response to receiving identification of the external stimulus from the user:
labeling the raw data file with the external stimulus identified by the user; and
storing the raw data file in a database;
at a second time succeeding the first time, receiving a search term from the user; and
in response to the search term matching the external stimulus:
retrieving the raw data file;
regenerating the visualization of the first timeseries of emotions; and
rendering the visualization for the user.

20. The method of claim 18, further comprising discarding timeseries biosignal data, from the rolling buffer, output by the set of biosensors during time periods preceding the fixed look-back duration.
